(19) United States
(12) Patent Application Publication
(10) Pub. No.: US 2017/0283755 A1
(43) Pub. Date: Oct. 5, 2017

(54) ELEMENT FOR INJECTING LIGHT HAVING AN ENERGY DISTRIBUTION

(71) Applicants: FRIEDERICH ALAIN LOUIS ANDRE, PARIS (FR); HUET HOLDINGS, PARIS (FR)

(72) Inventors: Alain FRIEDERICH, PARIS (FR); Gaël RUIZ, AYEN (FR); Mahmoud AFFI, NANTES (FR)

(21) Appl. No.: 15/529,064

(22) PCT Filed: Nov. 26, 2015
(86) PCT No.: PCT/EP2015/077849

§ 371 Date: May 23, 2017

(30) Foreign Application Priority Data
Nov. 26, 2014 (FR) 1461524

(51) Int. Cl. C12M 1/00; F21K 9/68; F21K 9/61; F21V 8/00; F21K 9/64

(52) U.S. Cl. CPC C12M 21/02; G02B 6/0096; F21K 9/64; F21K 9/61; F21K 9/68; C12M 31/08; C12M 31/10

(57) ABSTRACT

The invention relates to a light injector element (20) comprising a hollow body (21) extending according to a longitudinal axis (22), and a light source (23) placed facing an end (25) of the body (21), the light source (23) being configured to emit a light beam substantially parallel to the longitudinal axis (22) of said body (21), the injector element (20) further comprising at least one optical element (35i) arranged inside the body (21) and configured to let through a fraction of the light beam propagating in a central part (36i) of the body (21), and deflect towards the outside of said body (21) a fraction of the light beam propagating in a peripheral part (37i) of the body so as to locally distribute energy emitted by the light source (23).

The invention also relates to a photobioreactor (10) and a domestic lighting element comprising such a light injector element (20).

### TECHNICAL FIELD

The present invention relates to the general field of lighting, and in particular that of lighting for intensive and continuous culture of photosynthetic microorganisms.

### PRIOR ART

Many lighting elements are known from the prior art, such as for example luminescent or neon tubes, fluorescent tubes or light-emitting diodes (or LED).

In particular, an LED has an energetic emission diagram according to a Lambertian profile, that is, in the form of a lobe. An LED emits maximum energy flow in a principal direction perpendicular to its emission surface, and this energy flow decreases moving away from this principal direction.

Also, an LED has an emission cone whereof the solid angle is limited, typically by 90°. An LED therefore does not emit energy in directions having a strong inclination relative to the principal direction, especially beyond 45°. In this way, when an LED is installed for example in the ceiling of a room so as to emit light mainly to the vertical, it cannot illuminate at the horizontal, as a result reducing the quality of the lighting in the room. Such lighting quality can pose problems of comfort for a user and needs multiplication of systems of lighting to rectify this defect.

The use of LEDs however has considerable advantages, especially their substantial light output which is quasi-constant in the duration of use of the LED, in particular when the LEDs do not heat up.

As opposed to LEDs, fluorescent or neon tubes produce energy emission in all radial directions, even at the horizontal when installed as a ceiling light.

However, such lighting elements have light outputs much weaker than LEDs and their light intensity fades over time. Also, it often happens that such lighting elements scintillate, and can be particularly annoying for a user.

In the particular field of lighting for intensive culture of photosynthetic microorganisms, especially microalgae, it is essential that the energy flow emitted by the lighting elements is the most uniform possible in all directions of emission of said lighting element so as to improve the production output of said microalgae.

It is understood in fact that in general production depends directly on the quality of the lighting in the volume of the photobioreactor in which the microalgae are cultivated. It is necessary for all the biological liquid to be correctly lit with optimal average energy, which depends on the nature of the microalga.

Consequently, the interface between the light sources and the biological liquid has to be the biggest possible to maximise the useful volume of the biological liquid (bath).

In summary, it is evident that at concentrations d of the order of one gram per litre, the light is absorbed over a depth of A=0.5 cm. For a reactor of 1 m³, with a lighting surface of 1 m² (planar light source of 1 m²), the relevant volume of biological liquid will be only 1/200 m³. The ideal reactor would be such that the lit volume is equal to the volume of the reactor. More generally the quality factor of a reactor can be defined by the relation: Q=Sλ/V₀, where S is the lit surface (at the right power) in the volume V₀ of the reactor, and A the depth of penetration of the light.

With Ve being the volume of the illuminating elements dispersed in the reactor the mass production M can be expressed by the relation: M=(V₀−Vₑ)_{d} (where d is the mass of microalgae per unit of volume).

These two relations must be simultaneously maximised.

For this, document WO2011/080345 proposes for example light injector elements comprising a light guide of tubular form, at the end of which is placed an LED. The LED is enclosed by a mirror of parabolic or conical form or any other form which sends back the high-angle rays emitted by the LED in the axial direction of the injector.

Also, the light guide of the injector element is covered, at its end to the side of the LED, with a mirror whereof the opacity decreases moving away from the light source. In other words, this metallic mirror is full in the upper part of the injector element, becomes progressively semi-transparent, and finally disappears. In fact, without these mirrors, given the Lambertian energetic emission profile of the LED, the quantity of energy emitted by the tube along its side wall would decrease exponentially moving away from the LED, the consequence of which is that the light energy would essentially exit in the upper part of the injector element. It is understood therefore that using such mirrors is essential so that the injector element emits the most uniform energy possible along the tube.

This document proposes also placing a mirror at the end of the light guide opposite the LED so as to send back along the light guide of the injector element the light beams originating directly from the LED or reflected in directions having a low angle relative to the principal direction of emission, to compensate the increasing energy losses moving away from the LED. This mirror has for example a conical, half-spherical, or parabolic form, or even a more complex form.

Now, the use of such mirrors introduces significant absorption of the energy flow reflected by the mirrors, which also causes a loss of useful energy, causes local heating of the injector element and finally heating of the biological liquid (bath).

In fact, given a mirror of good quality and light emission of a wavelength of 0.8 μm, 5% of the light energy is absorbed during reflection on said mirror. In this way, if there is only a single reflection of light beams to be reoriented and if these beams represent for example 50% of the light flow, it is therefore 2.5% of the light energy which can be absorbed.

Now, especially in the case of the mirror enclosing the LED, the light beams having the strongest angles relative to the principal direction of emission are reflected several times. This effect is also reinforced for LEDs of large emission surface in comparison with the section of the injector element (surface of a few tens of mm²). Therefore, energetic absorption greater than 10% can be observed, and this even with a mirror of good quality.

The use of mirrors of conical form or even of more complex form limits the number of reflections of light beams and therefore reduces losses linked to absorption of reflected light flow.

However, apart from the fact that some of these mirrors can be industrially difficult to make, the absorption of the light flow they cause is considerable.

It is understood therefore that using such mirrors is particularly complicated and expensive in terms of power.

There is therefore a need to develop a light injector element for a photobioreactor for reducing light energy losses.

### Presentation of the Invention

An aim of the invention is therefore to propose a light injector element for reducing light energy losses between the energy emitted by the light source and the energy leaving the injector element. Another aim of the invention is to propose an injector element to provide uniform overall energy flow in all directions of emission of said injector element.

For this purpose, the invention proposes a light injector element comprising a hollow body extending according to a longitudinal axis, and a light source placed facing an end of the body,

the injector element being characterized in that the light source is configured to emit a light beam substantially parallel to the longitudinal axis of said body, and in that the injector element also comprises at least one optical element arranged inside the body and configured to let through a fraction of the light beam propagating in a central part of the body, and deflect towards the outside of said body a fraction of the light beam propagating in a peripheral part of the body so as to locally distribute the energy emitted by the light source.

According to other advantageous and non-limiting characteristics:
the optical element has an opening substantially coaxial with the longitudinal axis of the body so as to let through the fraction of the light beam propagating in the central part of the body;
the light injector element comprises a plurality of optical elements arranged inside the body, and extending at a distance from each other along said body, said optical elements being configured to let through a fraction of the light beam propagating in a central part of the body more and more reduced as the optical elements are moved away from the light source, so as to distribute energy emitted by the light source along the body;
the optical elements each have an opening substantially coaxial with the longitudinal axis of the body so as to let through a fraction of the light beam propagating in the central part of the body, said openings having a size decreasing with moving away relative to the light source;
the optical element(s) are diverging lenses, or diverging deflective prisms;
the light source comprises a plurality of vertical-cavity surface-emitting laser diodes, said plurality of diodes being arranged so as to form an emission surface substantially perpendicular to the longitudinal axis of the body;
the light source consists of vertical-cavity surface-emitting laser diodes, all configured to emit substantially equal wavelengths;
a phosphor is applied against a side wall of the body, the vertical-cavity surface-emitting laser diodes emit light at wavelengths preferably corresponding to blue light;
the light source comprises a first group of vertical-cavity surface-emitting laser diodes configured to emit light at wavelengths corresponding to red light, a second group of vertical-cavity surface-emitting laser diodes configured to emit light at wavelengths corresponding to blue light, and a third group of vertical-cavity surface-emitting laser diodes configured to emit light at wavelengths corresponding to green light, such that the injector element emits white light;
the light source is configured to emit more light in a peripheral zone than in a central zone of the emission surface;
the light source is configured to emit light only in the peripheral zone;
the central zone of the emission surface contains no diodes;
the light injector element further comprises a control unit configured to control the light source such that the peripheral zone of the emission surface emits more light than the central zone;
the light source is configured to emit non-uniform density of energy in the peripheral zone of the emission surface;
the diodes each have an elementary emission surface, and in which the elementary emission surfaces of the diodes of the peripheral zone have different dimensions to each other such that the light source emits a non-uniform energy density in the peripheral zone of the emission surface;
the injector element also comprises current injectors configured to deliver to the diodes a density of electric current or non-uniform voltage such that the light source emits a non-uniform energy density in the peripheral zone of the emission surface;
the optical elements are configured to deflect towards the outside of the body all the light emitted by the peripheral zone of the emission surface;
the injector element further comprises an end mirror arranged at one end of the body opposite the light source so as to send back to the body the part of the light beam being reflected against said end mirror;
the body has a cylindrical form, in particular cylindrical in revolution or parallelepiped;
the body has the form of a cylinder of revolution;
a mirror is applied against a part of the body corresponding to a half-cylinder so as to reflect towards the inside of the body the energy emitted towards said mirror;
the body has the form of a half-cylinder of revolution;
a mirror is applied against a planar side wall so as to reflect towards the inside of the body the energy emitted towards said mirror;
the emission surface of the light source has the form of a half-disc, and in which the light source is configured to emit a quantity of light energy decreasing in moving away from the straight line section of the emission surface in a direction extending perpendicularly to said straight line section;
the body substantially has the form of a rectangular parallelepiped;
the body comprises a first plate and a second plate, between which are placed at least one couple of optical elements, the optical elements of each couple being placed facing and at a distance from each other so as to form an opening substantially coaxial with the longitudinal axis of the body so as to let through the fraction of the light beam propagating in the central part of the body;
the body comprises a plate and a plane mirror placed facing each other, and at least one optical element placed at a distance from the plane mirror so as to form an opening substantially coaxial with the longitudinal axis of the body so as to let through the fraction of the light beam propagating in the central part of the body.

According to a second aspect, the invention relates to a photobioreactor intended for culture especially continuous culture of photosynthetic microorganisms, preferably microalgae, said photobioreactor comprising at least one culture container intended to contain the culture medium of the microorganisms, said photobioreactor being characterized in that it comprises a light injector element according to the first aspect of the invention, the body of said injector element being placed in the culture container.

According to a third aspect, the invention relates to a lighting element for domestic lighting, characterized in that it comprises a light injector element (20) according to the first aspect of the invention.

According to other advantageous and non-limiting characteristics, the lighting element also comprises a mirror placed facing the body so as to reflect the energy emitted towards said mirror.

### PRESENTATION OF FIGURES

Other characteristics, aims and advantages of the invention will emerge from the following description which is purely illustrative and non-limiting, and which must be considered in light of the appended drawings, in which:

FIG. 1 illustrates a schematic view, in vertical section, of a photobioreactor intended for culture especially continuous culture of photosynthetic microorganisms, preferably of microalgae, comprising a light injector element according to an embodiment of the invention;

FIG. 2 illustrates a schematic view, in vertical section, of a photobioreactor comprising a light injector element according to a variant of the embodiment illustrated in FIG. 1;

FIG. 3 illustrates a schematic view, in section, of a structure of a vertical-cavity surface-emitting laser (VCSEL) diode;

FIG. 4 illustrates a first example of emission profile of energy of a plurality of VCSEL in which the density of energy emitted is not uniform over the entire emission surface formed by the VCSELs;

FIG. 5 illustrates the distribution of energy emitted by the light injector element illustrated in FIG. 4 over its entire length, when the VCSELs have an emission profile such as illustrated in FIG. 4;

FIG. 6 illustrates a second example of emission profile of energy of a plurality of VCSELs in which the density of energy emitted is not uniform over the entire emission surface formed by the VCSELs;

FIG. 7 illustrates the distribution of energy emitted by the light injector element illustrated in FIG. 6 over its entire length, when the VCSELs have an emission profile such as illustrated in FIG. 6;

FIG. 8 illustrates a schematic view, in cross-section, of a lighting element comprising a light injector element according to a first embodiment of the invention;

FIG. 9 illustrates a schematic view, in cross-section, of a lighting element comprising a light injector element according to a second embodiment of the invention;

FIG. 10 illustrates a schematic view, in cross-section, of a lighting element comprising a light injector element according to a third embodiment of the invention;

FIG. 11 illustrates a schematic view, in perspective, of the lighting element illustrated in FIG. 10;

FIG. 12 illustrates a perspective view, in vertical section, of a photobioreactor comprising a light injector element according to a variant of the embodiments illustrated in FIGS. 1 and 2;

FIG. 13 illustrates a perspective view, in vertical section, of a photobioreactor comprising a lighting element comprising a light injector element according to a fourth embodiment of the invention.

### DETAILED DESCRIPTION

### Case of Lighting for Intensive and Continuous Culture of Photosynthetic Microorganisms

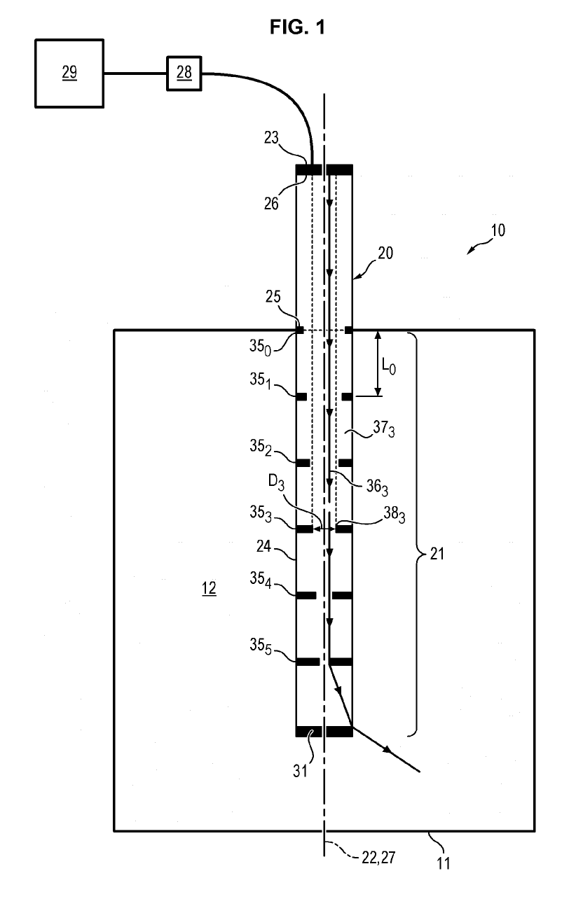

FIG. 1 shows a photobioreactor 10 intended for culture especially continuous culture of photosynthetic microorganisms, preferably of microalgae, according to an embodiment of the invention.

The photobioreactor 10 comprises at least one culture container 11 intended to contain the culture medium 12 of microorganisms, and at least one light injector element 20.

The light injector element 20 comprises a cylindrical and hollow body 21 extending according to a longitudinal axis 22. When used in a photobioreactor, the longitudinal axis 22 of the light injector element 20 coincides substantially with a vertical direction.

Cylinder means the volume generated by translation of a surface (forming a base) according to a direction orthogonal to the surface. For example, the body 21 can have the form of a cylinder of revolution (cylinder whereof the base is a disc) or a prism (cylinder whereof the base is a polygon). In particular, the body 21 can have the form of a rectangular parallelepiped.

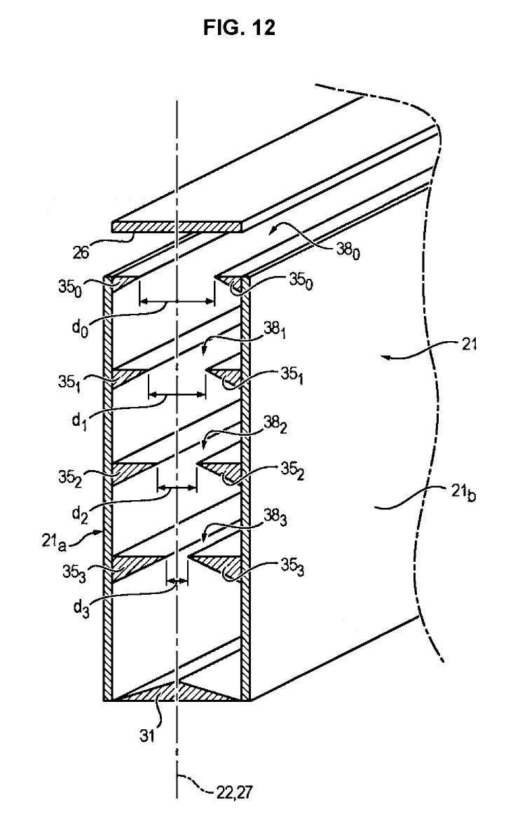

The body 21 is placed in the culture container 11. The body 21 can have the form of a cylinder of revolution or of a prism. In the case of a body 21 of the form of a rectangular parallelepiped, as illustrated in FIG. 12, two opposite faces of said body 21 are preferably plates 21a, 21b placed close to each other. The plates 21a, 21b define the length (height) and the width of the body 21, whereas the distance between the plates 21a, 21b defines the thickness of the body 21. The plates are for example made of Poly(methyl methacrylate) (PMMA) or glass.

The body 21 of the light injector element 20 is coupled with a light source 23 (arranged at the top end of the light injector element 20 when the latter is oriented vertically) to guide the flow of light emitted by the light source 23 and transmit it to the culture medium 12 via its side wall(s) 24. This coupling is for example via one optical element 35i (in particular diverging or converging lens) configured to deflect the light beam, as will be explained hereinbelow. The index step between the central cavity and the envelope of the body 21 defining the side walls 24 (plates 21a, 21b for a parallelepiped body) controls lateral transmission of light.

In the case of a body 21 in the form of a rectangular parallelepiped, as illustrated in FIG. 12, light is emitted laterally through the plates 21a, 21b. Preferably and for reasons of managing thermal losses, the light source 23 is placed outside the culture container 11, facing a proximal end 25 of said body 21, especially in contact with a radiator (preferred common to all injector elements) refrigerated by coolant.

It is understood that the present light injector element 20 transfers light energy from the source 23 to the side wall only by refraction phenomena, that is, deflection of light beams to interfaces between two media (i.e. index steps), irrespectively of whether at the level of the optical elements 35i of lens type, the side wall 24, or any other optical elements (see below).

So-called diffusion phenomena (deflection of light beams by particles in a heterogeneous medium) are as such best avoided (in a given medium transparency maximal is favoured). This loses almost no energy in the medium and restores 100% of the energy supplied by the source 23. Diffusing media in fact tend to heat under the effect of radiation.

The light source 23 is configured to emit a light beam substantially parallel to the longitudinal axis 22 of the body 21, and can for example consist of one or more laser sources, as shown below.

The injector element 20 further comprises at least one optical element 35i arranged inside the body 21 and configured to let through a fraction of the light beam propagating in a central part 36i of the body 21, and deflect towards the outside of the body 21 a fraction of the light beam propagating in a peripheral part 37 of the body 21. In this way, the optical element 35i locally distributes the energy emitted by the light source 23. In other words, the optical element 35i on the light beam takes a fraction of energy to deflect it towards the outside of the body 21.

Preferably, as illustrated in FIG. 1, the injector element 20 comprises a plurality of optical elements 35i arranged inside the body 21 at a distance from each other along said body 21, the optical elements 35i also being configured to let through a fraction of the light beam propagating in a central part 36i more and more reduced as the optical elements 35i are moved away from the light source 23. In this way, each time the light beam passes through an optical element 35i, the latter takes some of its energy to deflect it towards the outside of the body 21. The optical elements 35i therefore distribute the energy of the light beam along the body 21.

It is understood that it is possible to remove the energy emitted by the light source 23 so as to distribute it uniformly along the body 21, such that the average energy along said body 21 is sufficient to allow development of microorganisms.

The energy emitted along the body 21 is especially between a predetermined energy threshold and so-called saturation energy of microorganisms. The energy threshold corresponds to the minimal energy necessary to initiate photosynthesis.

The optical elements 35i are preferably of the same form and substantially the same dimensions as the cross-section of the body 21, the edge of the optical elements 35i being placed against the inner surface of the side wall of the body 21. In this way, in the case of a body 21 of circular cross-section, the optical elements 35i have a diameter substantially equal to the diameter of the body 21, whereas in the case of a body 21 of the form of a rectangular parallelepiped the optical elements 35i have a length and width substantially equal to the width and thickness of the body 21, respectively.

For example, the optical elements 35i are “holed”, they have an opening 38i substantially coaxial with the longitudinal axis 22 of the body 21 so as to let through only that fraction of the light beam propagating in the central part 36i of the body 21 without deflecting it. The openings 38i are also smaller and smaller as the optical elements 35i are moved away from the light source 23.

The opening 38i of the optical elements 35i preferably has the same form as the cross-section of the body 21. In this way, when the body 21 is tubular the opening 38i of the optical elements 35i is preferably circular, the diameter Di of the openings 38i being smaller and smaller as the optical elements 35i are moved away from the light source 23.

The optical elements 35i are for example diverging lenses or deflecting prisms, especially annular prisms. The lenses 35i can have an identical or different focal length. Similarly, the prisms 35i can have identical or different geometries.

When the body 21 is tubular, each lens 35i is positioned for example in said body by means of an elastic ring (not shown) made of plastic, stuck against the inner wall of the body 21.

In the example illustrated in FIG. 1, the injector element 20 is tubular and the optical elements 35i are diverging lenses having an opening 38i of diameter Di smaller and smaller as the lens 35i is moved away from the light source 23. In these examples, when the light source 23 emits the light beam in the direction of emission, a lens 35i intercepts a fraction of the light beam and deflects it towards the outside of the body 21. The lens 35i therefore outputs an average energy of the body over a length Li dependent on the focal length fi of the lens 35i and its diameter Di. The fraction of the light beam intercepted by the lens 35i determines the energy injected over the length Li. At the end of the length Li, a new fraction of the light beam is intercepted by a lens 35i+1 (to the extent where the lens 35i+1 has an opening 38i+1 of diameter Di+1 less than the lens 35i) and is deflected towards the outside of the body 21 over a length Li+1 dependent on the focal length fi+1 of the lens 35i+1 and its diameter Di+1. The power received by the lens 35i+1 is proportional to the difference in surfaces between the openings 38i and 38i+1. It is understood that carrying out this operation n times (that is, by positioning n lenses 35i in the body) enables progressive removal of the energy of the light beam to distribute it uniformly over the entire length of the body 21.

The length Li corresponds to the distance between the lens 35i and the point of attack of the fraction of the light beam deflected by the edge of the opening 38i of the lens 35i onto the side wall 24 of the body 21. It is understood that to distribute energy uniformly over the entire length of the body 21, the lens 35i+1 is preferably placed at a distance from the lens 35i corresponding to the length Li.

It is understood also that to achieve distribution of the energy uniform over the entire length of the body 21 the parameters of each lens 35i are optimises as a function of the number n of lenses 35i. These parameters are the following: the diameter Di, the length Li (or distance between two consecutive lenses 35i and 35i+1), and the focal length fi of each lens 35i. It is also clear that optimisation of the parameters of the lenses 35i can also take into account, for photosynthetic microorganism growth, the fact that the average energy emitted by the body 21 must be between the energy threshold and the so-called saturation energy of the microorganisms.

The injector element 20 progressively punctures the energy conveyed in the light beam and deflects it towards the outside of the body 21 in a controlled way.

As a variant, in the particular case of a body 21 of the form of a rectangular parallelepiped, as illustrated in FIG. 12, the openings 38i can be formed by couples of deflecting prisms 35i placed facing and at a distance from each other. Each prism 35i of a couple of prisms has a first edge placed against the inner surface of a plate 21a, 21b opposite the body 21, and a second edge extending facing and at a distance di from the second edge of the other prism 35i of the couple of prisms, the distance di between the prisms 35i of each couple forming the opening 38i. The distance di is smaller and smaller as the optical elements 35i are moved away from the light source 23.

In the example illustrated in FIG. 1, the injector element 20 further comprises a mirror 31 arranged at a distal end of the body 21, i.e. an end opposite the light source 23. The end mirror 31 is configured to send back the light beam in the body 21 so as to compensate the loss of energy extracted from the body 21 when moving away from the light source 23. The end mirror 31 makes the energy flow emitted by the side wall 24 of the body 21 more uniform. The end mirror 31 has for example a plane reflecting surface, half-spherical, conical or parabolic. Preferably, the profile of the reflecting surface of the mirror 31 is determined such that the light energy reflected by the end mirror 31 decreases moving more closely to the light source 23 so as to reduce to a maximum the energy returning to the light source 23. It is clear in fact that to limit energy losses in the injector element 20 it is advantageous to send back to the body 21 the fraction of the light beam arriving directly at the end mirror 31 (that is, without having been reflected by the side wall 24 of the body 21) and the light flow reflected by the side wall 24 of the body 21 arriving at the end mirror 31. It is understood also that, always to limit the energy losses in the injector element 20, it is advantageous to reduce the fraction of the light beam returning to the light source 23 especially to prevent the latter from heating up and that some of the energy emitted is not transmitted to the culture medium 12. The mirror 31 preferably has the same dimensions as the cross-section of the body 21.

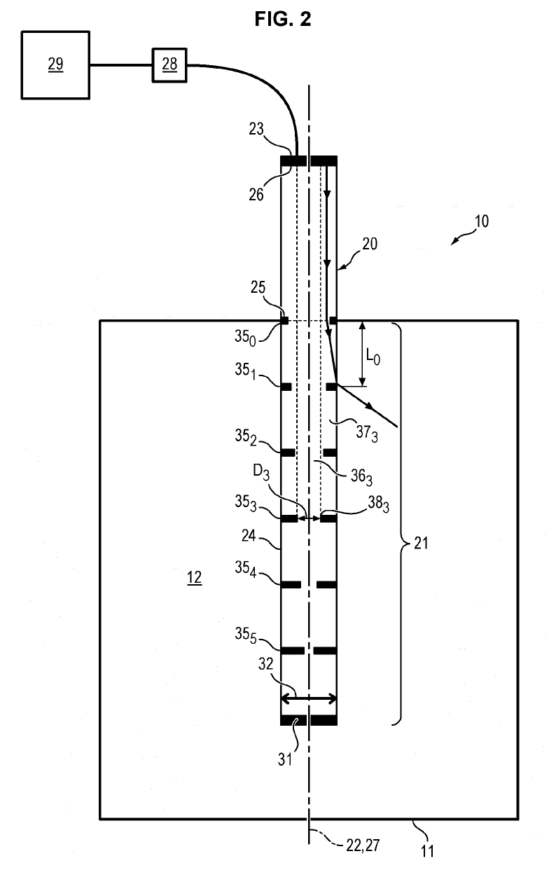

As illustrated in FIG. 2, the injector element 20 can also be fitted with a diverging or converging end lens 32 arranged inside the body 21 facing the end mirror 31 so as to boost the angle of attack against the side wall 24 of the body 21 of the fraction of the light beam reflected against the end mirror 31, and the lens and mirror couple of form adapted must be optimised for this reason. In this way, the energy reflected by the end mirror 31 is more rapidly consumed and the risks that this energy might not return to the light source 23 are limited.

According to a preferred embodiment, the light source 23 comprises one or more laser sources, in particular a plurality of vertical-cavity surface-emitting laser diodes, called VCSEL, arranged so as to form an emission surface 26 substantially perpendicular to the longitudinal axis 22 of the body 21 and emit a light beam in a direction of emission 27 substantially parallel to the longitudinal axis 22 of the body. The VCSELs are fed with electric current by means of at least one power supply 28. The power supply or the power supplies 28 are for example controlled by a control unit 29. The emission surface 26 is preferably centred on the (end 25) of the body 21. The emission surface 26 preferably has a form adapted to the cross-section of the body 21. In this way, in the case of a body 21 having a circular cross-section, the emission surface 26 will preferably be a disc, whereas in the case of a body 21 of the form of a rectangular parallelepiped, the emission surface 26 will preferably be a band, as illustrated in FIG. 12.

The VCSELs are solid lasers with direct gap semiconductor for producing emission of coherent light, contrary to LEDs which generate incoherent light only.

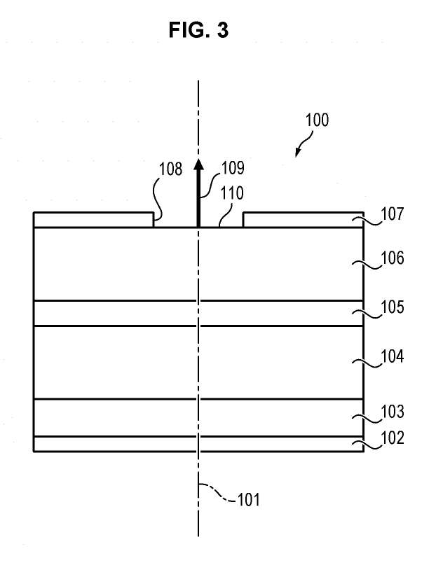

As illustrated in FIG. 3, a VCSEL comprises a stacked layer structure 100 according to the direction of emission 101 of the light beam. The structure 100 comprises especially:
a so-called lower metallic contact layer 102,
a semiconductor substrate 103 having n-type doping,
a so-called Bragg mirror 104 having n-type doping,
at least one quantum well 105 forming the resonating vertical cavity,
a so-called upper Bragg mirror 106 having p-type doping,
a so-called upper metallic contact layer 107 having an opening 108, in which a transparent and conductive metallic oxide layer is deposited, and by which the light beam 109 is emitted.

A VCSEL therefore emits a light beam via an elementary emission surface 110 substantially perpendicular to the stacking direction of the layers 102 to 107, as opposed to conventional solid lasers which emit via the tranche, that is, via a surface substantially parallel to the stacking direction of the layers (flank of the cavity).

The elementary emission surface of a VCSEL is for example of the order of a hundred μm² and the optical power supplied exceeds several tens of milliwatts in the field of the visible for an emission surface of a few hundred μm².

The fact that VCSELs have a structure 100 in layers extending perpendicularly to the direction of emission 101 (technology known as “planar”) connects a large number (a few hundred) on a millimeter surface to form a C-VCSEL “integrated laser circuit” comprising a number N of VCSELs. The light energy emitted by the C-VCSEL is the sum of the light energy emitted by each elementary VCSEL if there is no coupling between VCSEL, especially via the semiconducting layers 103 to 106. A C-VCSEL produces light emissions of high power with almost zero divergence, as opposed to LEDs. A C-VCSEL for example produces powers exceeding tens of optical watts per mm².

The plurality of VCSELs of the light source 23 is organised into C-VCSEL such that all of the elementary emission surfaces 110 of the VCSELs form the emission surface 26.

It is understood that use of a C-VCSEL transports the light energy over the entire length of the body 21 as well as doing without mirrors which in the prior art were necessary for correcting the Lambertian energy profile of the LEDs, as a result reducing energy losses which were linked to use of these mirrors, and manufacturing costs of the injector element 20.

As will be evident later, the C-VCSEL can be configured advantageously to exhibit variable density of energy over its emission surface 26. The skilled person knows a plurality of techniques for arriving at this result, and the present light injector element will not be limited to any of them.

In particular, the complex structure of a VCSEL (Bragg mirrors, active layers, etc.) is made by epitaxy (epitaxy by molecular jets for example) on a conductive substrate 103 of at least the entire surface of the C-VCSEL. Delimitation of the elementary VCSELs (that is, of the elementary emission surface of each VCSEL) is done by optical lithography. It is possible by means of “optical masks” to define the dimensions of the elementary emission surface 110 of each VCSEL and their surface densities (in other words vary the pitch between two adjacent VCSELs) on a given zone of the C-VCSEL. Connection technologies form the subject matter of deposits through masks adapted to the needs of electric controls, well known to the skilled person. It is possible to provide “holes” in the emission surface 26, in other words zones devoid of VCSEL. For clarity of description, any zones having zero light emission but enclosed by zones having non-zero light emission will be considered as forming part of the emission surface 26.

Alternatively, in the C-VCSEL, each VCSEL can be individually connected to a power supply 28. In this case, the control unit 29 can be configured to individually control the power supplies 28 to deliver different current densities according to the VCSEL. The voltage of the VCSELs can also be controlled. The C-VCSEL can also be delimited by zones and the VCSELs of each zone can be connected together and to a power supply 28 dedicated per zone. In these two latter cases, the control unit 29 is for example a matrix control circuit. The VCSELs can on the contrary be connected together and to a single power supply 28. In this case, the power supply 28 is controlled by the control unit 29 so as to deliver uniform current or voltage density (in other words, if the VCSELs have the same impedance per surface unit, the control voltage is the same on all VCSELs). Advantageously, the light source 23 is configured to emit more light in a peripheral zone 33 than in a central zone 34 of the emission surface 26. The central zone 34 of the emission surface 26 preferably emits no light. In this way, the part of the light beam reflected directly (that is, without having been reflected by the side wall 24 of the body 21) against the end mirror 31 is limited or even eliminated, accordingly reducing the energy reflected by the end mirror 31 directly towards the light source 23. This also limits the quantity of energy reflected by the end mirror 31 and therefore reduces the energetic losses linked to this reflection.

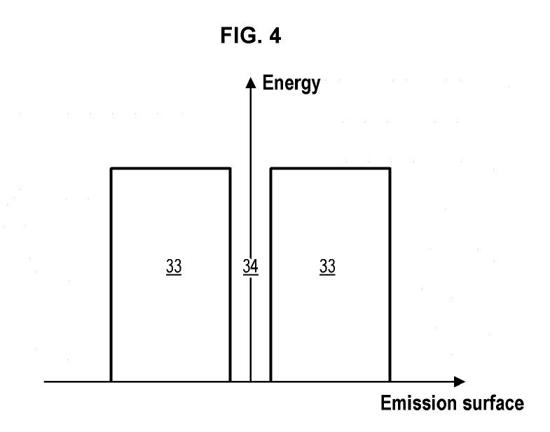

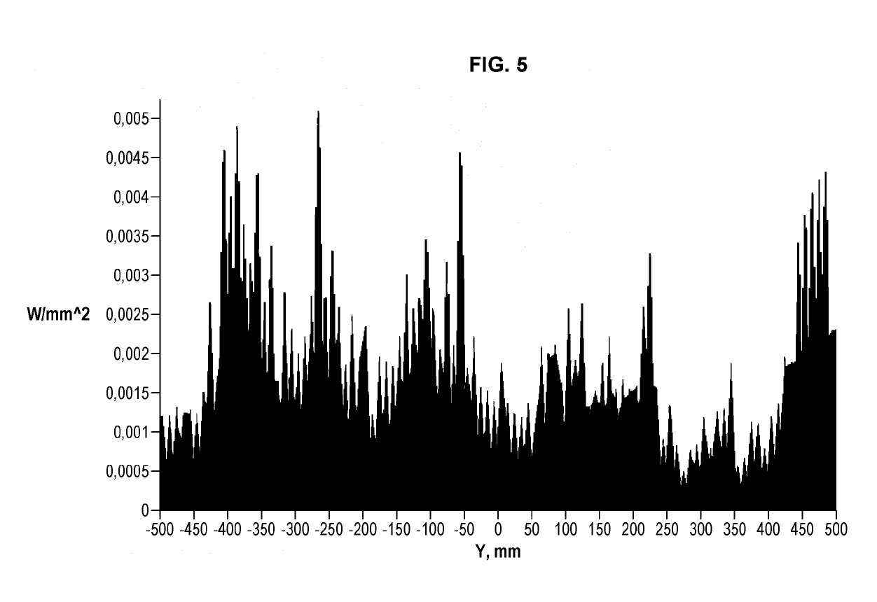

An example of emission profile of the light source 23 having such density of energy emitted by the emission surface 26 is illustrated in FIG. 4. In this example, the density of energy is zero in the central zone 34 and uniform in the peripheral zone 33. In this example, the body 21 is a cylinder of revolution and the emission profile is rotationally-symmetrical about the longitudinal axis 22 of the body 21. The central zone 34 of the emission surface 26 has the form of a disc and the peripheral zone 35 of the emission surface 26 has the form of a ring. FIG. 5 further illustrates the distribution of the energy emitted by the injector element 20 over the entire length of the body 21, when the VCSELs have such an energy emission profile. This figure shows that the injector element 20 emits an overall uniform level of energy all along the body 21.

According to this preferred embodiment, the central zone 34 of the emission surface 26 comprises for example no VCSEL. The substrate treated by photolithography can also be configured to deactivate the VCSELs (the elementary emission surfaces of the VCSELs) of the central zone 34, such that only the VCSELs of the peripheral zone 33 emit the light.

According to a variant, the control unit 29 regulates the light source 23 such that the peripheral zone 33 of the emission surface 26 emits more light than the central zone 34. For this, the control unit 22 for example commands the power injector(s) 28 connected to the VCSELs of the central zone 34 to deliver low or even zero current density, and the power injector(s) 28 connected to the VCSELs of the peripheral zone 33 to deliver a stronger current density. The VCSELs of the central zone 34 are preferably extinguished. The VCSELs can also be voltage-controlled.

Advantageously, the optical elements 35i are configured to deflect towards the outside of the body 21 all the light emitted by the peripheral zone 33 of the emission surface 26. For this, the central zone 34 of the emission surface 26 has dimensions greater than or equal to those of the opening 38i of the optical element 35i the farthest from the laser source 23. It is understood in fact in this case that the whole light beam is deflected by the optical elements 35i and that no fraction of the light beam is reflected directly against the end mirror 31 without having been previously deflected. This prevents the end mirror 31 reflecting the light beam directly onto the light source 23, which would cause energy losses and overheating of said light source 23.

Advantageously, the light source 23 is further configured to emit non-uniform density of energy in the peripheral zone 33 of the emission surface 26. For this, the substrate (after deposit of layers defining the structure 100 illustrated in FIG. 2) treated by photolithography can be configured to modulate the elementary emission surface of the VCSELs of the peripheral zone 33 of the emission surface 26 to obtain non-uniform density of energy (in the C-VCSEL). As a variant, the control unit 29 commands the power supplies 28 to deliver non-uniform current density to the peripheral zone 33 of the emission surface 26.

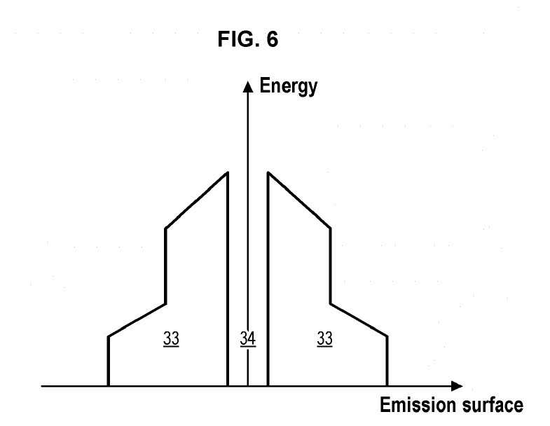

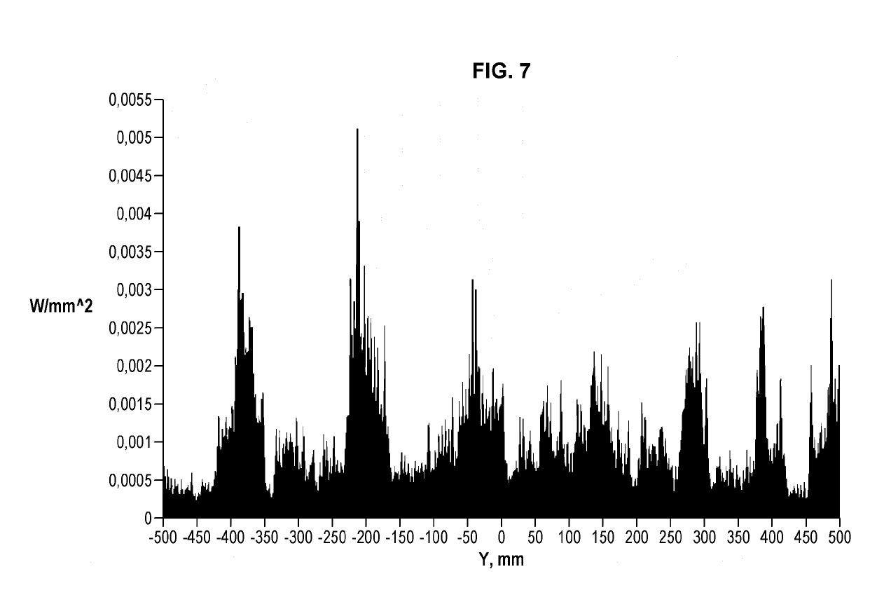

An example of emission profile of the C-VCSEL having such density of energy in the peripheral zone 33 of the emission surface 26 is illustrated in FIG. 6. In this example, the body 21 is a cylinder of revolution and the emission profile is rotationally-symmetrical about the longitudinal axis 22 of the body 21. FIG. 5 shows that the light source 23 is configured to emit energy decreasing from the edge of the central zone 34 towards the edge of the emission surface 26. More precisely, on a first zone extending from the edge of the central zone 34 the energy decreases moving away from the central zone 34 moving from a high level of energy to an average high level of energy, then on a second zone extending from the edge of the first zone towards the edge of the emission surface 26 the energy decreases again moving away from the central zone 34 moving from a low average level of energy to a low level of energy. At the interface between the first zone and the second zone the level of energy is therefore discontinuous. FIG. 7 further illustrates the distribution of energy emitted by the injector element 20 over the entire length of the body 21, when the VCSELs have such an energy emission profile. It is clear from comparing this figure to FIG. 5 that the emission profile illustrated in FIG. 6 further improves the uniformity of the distribution of the energy emitted by the injector element 20 along the body 21. Similar results are obtained with an injector element 20 such as illustrated in FIG. 12, the latter having an emission profile overall uniform over the entire surface of the plates 21a, 21b.

The fact of using a C-VCSEL as light source 23 in combination with the optical elements 35i further creates injector elements of considerable length, greater than one meter (as in the cylindrical body 21 illustrated in FIGS. 1 and 3) or of large surface (as in the body 21 of the form of a rectangular parallelepiped illustrated in FIG. 12) and which has a particularly high output (power transferred to the culture medium/power emitted by the C-VCSEL), especially greater than 90%.

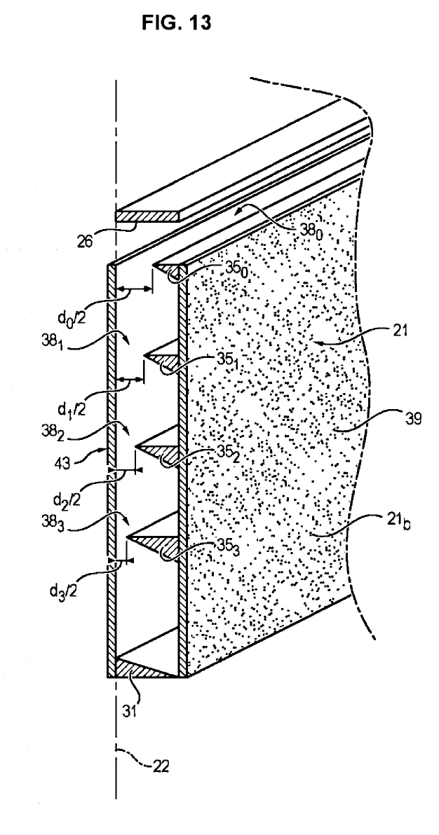

In the examples illustrated in FIGS. 1 and 2, the emission surface 26 of the C-VCSEL is substantially of the same dimensions as the cross-section of the body 21. As a variant, as illustrated in FIGS. 12 and 13, the emission surface 26 can also be of dimensions less than the cross-section of the body 21. In the latter case, the injector element 20 can also be provided with an optical system projecting an enlarged image of the C-VCSEL, preferably of the section of the optical guide, on the diverging lens (or the prism) 35₁ located at the input of the body 21. This device well known to the skilled person comprises at least two lenses or two prisms.

The control unit 29 can also be configured to control the light source 23 such that it emits pulsed light. In particular, with the VCSELs, the light can be modulated at high frequencies, especially beyond GHz. On the contrary, the LEDs may possibly go beyond 100 MHz.

The injector element 20 can also be attached to a planar heat pipe configured to recover thermal losses from the light source 23. The planar heat pipe is placed in contact with the light source 23, outside the culture container 11. In this way, the temperature of the culture container 11 is more easily kept at an ad hoc temperature for growth of photosynthetic microorganisms.

It is evident that for use in a photobioreactor, the light source 23 (or the C-VCSEL) is configured to emit wavelengths corresponding to red light, especially from 620 to 780 nm.

### Case of Domestic Lighting, Especially of Injector Elements of White Light

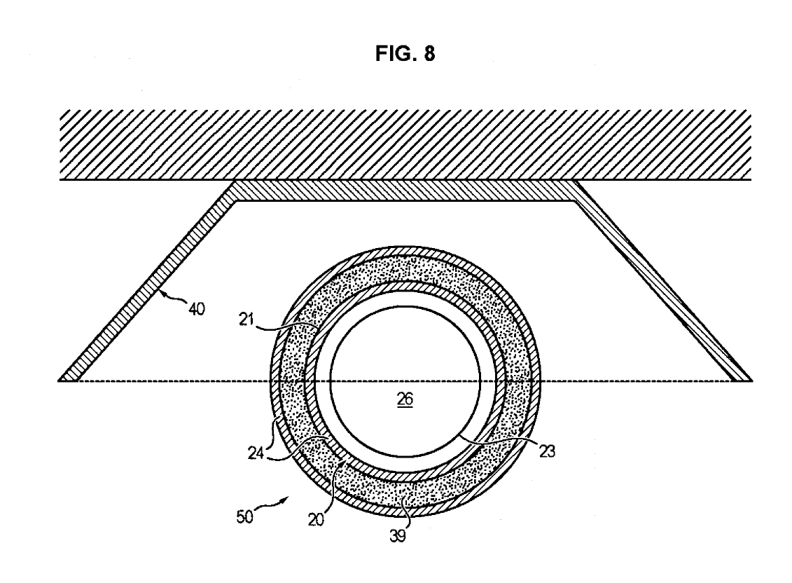

FIGS. 8, 9, 10, 11 and 13 show a lighting element 50 for domestic lighting according to different embodiments of the invention.

The lighting element 50 comprises a light injector element 20 such as previously described.

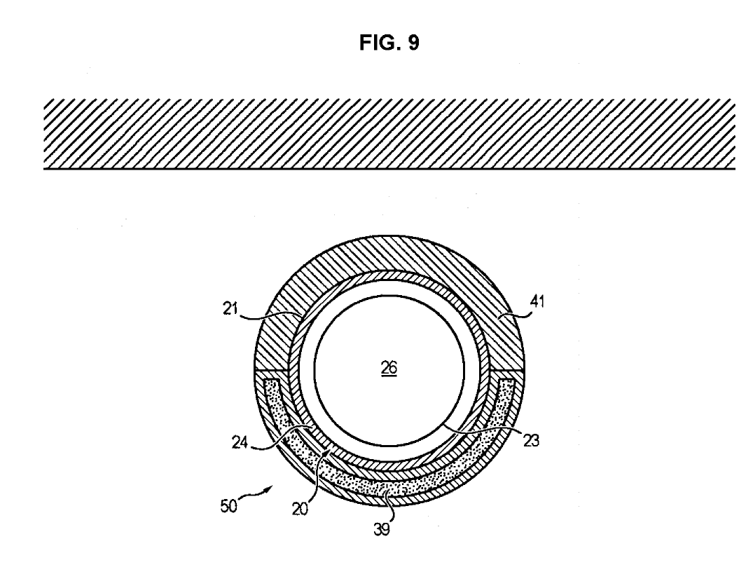

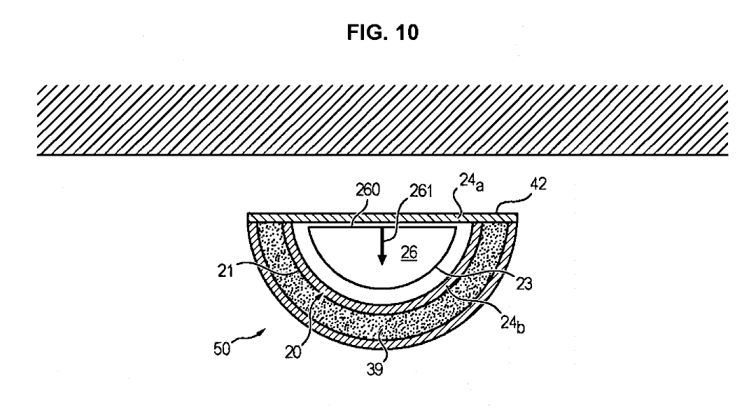

For usage of the injector element 20 for domestic lighting, the injector element 20 comprises for example a phosphor 39 applied along the side wall of the body 21. The phosphor 39 is for example protected by encapsulation in transparent organic or mineral material, as is for example illustrated in FIG. 9. The body 21 can also have a double wall 24 between which the phosphor 39 is arranged, as is for example illustrated in FIGS. 8 and 10. To produce an injector element 20 emitting white light, the phosphor 39 is a mixture of three different phosphors (Red Green Blue or RGB) and the light source 23 (or the C-VCSEL) is configured to emit wavelengths corresponding to blue light, especially from 446 to 500 nm.

It is clear that the conversion process of blue light into white light by phosphor cancels out the directional character of the light. In other words, the primary blue light is directional (laser) whereas light emitted by the phosphor is diffused. The latter cannot propagate in the light guide and easily be configured to obtain homogeneous flow at the outer surface of the injector.

As a variant, when the light source 23 comprises a C-VCSEL, the C-VCSEL comprises a first group of VCSELs configured to emit wavelengths corresponding to red light, especially from 620 to 780 nm, a second group of VCSELs configured to emit wavelengths corresponding to blue light, especially from 446 to 500 nm, and a third group of VCSELs configured to emit wavelengths corresponding to green light, especially from 500 to 578 nm. For this, it is for example possible to carry out localised epitaxies to obtain the first, second and third groups of VCSELs, and to fit them into each other so as to preferably have at any point of the C-VCSEL subgroups of VSCELs comprising a red VSCEL, a green VSCEL, and a blue VSCEL. It is clear that according to this variant beams of red, blue and green colour are emitted by the C-VCSEL and are then mixed in the body 21 of the injector element 20 to produce emission of white light towards the outside of the injector element 20.

The following embodiments were created for use of the injector element 20 as a ceiling light.

According to a first embodiment illustrated in FIG. 8, the body 21 of the injector element 20 has the form of a cylinder of revolution and the lighting element 50 further comprises a mirror 40 placed facing and at a distance from the body 21 so as to reflect the white light emitted by the injector element 20 towards the rear (the ceiling) in a forward direction (the floor of the room).

The mirror 40 extends for example according to a longitudinal axis parallel to the longitudinal axis 22 of the injector element 20 and has a cross-section substantially in an inverted U. For this the mirror 40 comprises a first panel placed parallel to the ceiling and second and third panels extending on either side of the first panel to form with said first panel an angle of around 120°. It is evident that according to this embodiment the injector element 20 emits light over its entire circumference (2π).

According to a second embodiment illustrated in FIG. 9, the body 21 of the injector element 20 has the form of a cylinder of revolution and, on a part of the body 21 corresponding to a half-cylinder, the phosphor is replaced by a mirror 41 having a reflecting surface facing the inside of the body 21 so as to reflect towards the inside of the body 21 the energy emitted towards the mirror 41. The part of the body 21 receiving the mirror 41 is intended to be placed facing the ceiling so as to reflect towards the inside of the body 21 the energy emitted by the injector element 20 towards the ceiling. It is clear that according to this embodiment, the injector element 20 emits light over a half-circumference (π).

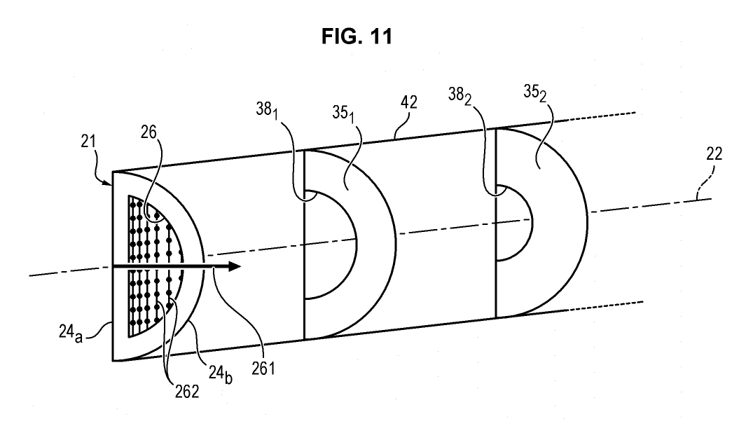

According to a third embodiment illustrated in FIGS. 10 and 11, the body 21 of the injector element 20 has the form of a half-cylinder of revolution whereof the planar side wall 24a is provided with a plane mirror 42 so as to reflect towards the inside of the body 21 the energy emitted towards the mirror 42, and the convex side wall 24b is provided with a phosphor 39. The planar side wall 24a is intended to be placed facing the ceiling so as to reflect towards the inside of the body 21 the energy emitted by the injector element 20 towards the ceiling. A phosphor 39 can for example be deposited against the outer surface of the convex side wall 24b, then encapsulated to protect it from the outside environment. It is clear that according to this embodiment, the injector element 20 emits light over a half-circumference (π).

According to this embodiment, the emission surface 26 of the C-VCSEL preferably has the form of a half-disc, the straight line section 260 of the half-disc being arranged parallel to the planar side wall 24a of the body 21 but without touching it.

According to this embodiment, the C-VCSEL is preferably further configured to compensate the loss of energy density received at ground level when moving away perpendicularly to the longitudinal axis 22 of the injector element 20, from its projection to the ground. For this, the surface density of VCSEL is increased when a shift is made to a line perpendicular to the straight line section 260 of the injector, in moving more closely to this straight line section 260. The function in variation of the surface density of VCSEL preferably has quadratic dependence, linked to the distance between the longitudinal axis 22 of the injector element 20 and the relevant lit point on the ground.

Otherwise expressed, the C-VCSEL is configured to emit a quantity of light energy decreasing moving away from the straight line section 260 of the emission surface 26 in a direction 261 extending perpendicularly to said straight line section 260. For this, the VCSELs can for example be aligned parallel to the straight line section 260 of the emission surface 26, the distance between two adjacent lines 262 of VCSEL increasing in moving away from the straight line section 260 of the emission surface 26 in the direction 261. The increase in surface density of VCSEL in the C-VCSEL increases for example quadratically in moving more closely to the straight line section 260 of the emission surface 26 in the direction 261. According to this particular embodiment, the VCSELs can have in the C-VCSEL an elementary emission surface of the same dimensions. Alternatively, the elementary emission surface of the VCSELs can be decreased in moving away from the straight line section 260 in the direction 261. It is clear that this quadratic increase of the density of VSCEL when shifting in the direction of the right edge of the C-VCSEL circuit (in direction 261) keeps constant the energy density arriving at the ground, when a shift is made to the ground perpendicularly to the axis 22 of the injector. The application of this correction of the flow arriving at the ground is limited by the maximal density of VCSELs implanted in the C-VCSEL. This technique significantly enlarges the lighting field perpendicularly to the direction 22.

According to this embodiment, the optical elements 35i and their opening 38i preferably have a form of hemi-lenses holed at their centres to distribute the energy of the light beam between the elements 35ᵢ. The straight line section of the hemi-lenses is arranged against the planar side wall 24a of the body. In this case, the injector element 20 could be produced by positioning the optical elements 35i in the body 21, then reclosing the body 21 using the mirror 42, the latter acting as a cover.

According to a fourth embodiment illustrated in FIG. 13, the injector element 20 corresponds to a semi-injector element 20 as illustrated in FIG. 12. Otherwise expressed, the first plate 21a and the prism 35i of each couple of prisms 35i associated with said first plate 21a are replaced by a plane mirror 43 placed facing the second plate 21b so as to reflect towards the inside of the body 21 the energy emitted by the injector element 20 towards the mirror 43. The mirror 43 is placed at a distance di/2 from the second edge of the prisms 35i. The mirror 43 is intended to be placed facing the ceiling so as to reflect towards the inside of the body 21 the energy emitted by the injector element 20 towards the ceiling. A phosphor 39 can for example be deposited on the outer surface of the second plate 21b, then encapsulated to protect it from the outside environment. It is clear that according to this embodiment the injector element 20 emits light over the entire surface of the second plate 21b.

The injectors described in FIGS. 8 and 12 described hereinabove can also be applied in the event of lighting for intensive and continuous culture of photosynthetic microorganisms. In this case the lighting elements 50 will contain no phosphor 39 and the light source 23 (or the C-VCSEL) will be configured to emit wavelengths corresponding to red light, especially from 620 to 780 nm. The injectors described in FIGS. 8, 9, 10, 11, 12 and 13 can be used for ceiling or wall lighting. In the versions described above where phosphors are used, it must be noted that it is possible according to careful choice of the composition of the phosphors to produce lighting of diverse colours. Similarly for the RGB C-VCSEL version, a change in the relative weight of the light intensities emitted by each of the red, green or blue groups modifies the colour of the light emitted by the injector elements 10.

1. A light injector element (20) comprising a hollow body (21) extending according to a longitudinal axis (22), and a light source (23) placed facing an end (25) of the body (21),
the injector element (20) being characterized in that the light source (23) is configured to emit a light beam substantially parallel to the longitudinal axis (22) of said body (21), and in that the injector element (20) further comprises at least one optical element (35i) arranged inside the body (21) and configured to let through a fraction of the light beam spreading in a central part (36i) of the body (21), and deflect towards the outside of said body (21) a fraction of the light beam spreading in a peripheral part (37i) of the body, so as to locally distribute energy emitted by the light source (23).

2. The injector element (20) according to claim 1, wherein the optical element (35i) has an opening (38i) substantially coaxial with the longitudinal axis (22) of the body (21) so as to let through the fraction of the light beam spreading in the central part (36i) of the body (21).

3. The injector element (20) according to one of claims 1 and 2, comprising a plurality of optical elements (35i) arranged inside the body (21), and extending at a distance from each other along said body (21), said optical elements (35i) being configured to let through a fraction of the light beam spreading in a central part (36i) of the body (21) more and more restricted as the optical elements (35i) are moved away from the light source (23) so as to distribute energy emitted by the light source (23) along the body (21).

4. The injector element (20) according to claim 3, wherein the optical elements (35i) each have an opening (38i) substantially coaxial with the longitudinal axis (22) of the body (21) so as to let through a fraction of the light beam spreading in the central part (36i) of the body (21), said openings (38i) having a size decreasing when moving away relative to the light source (23).

5. The injector element (20) according to one of claims 1 to 4, wherein the optical element(s) (35i) are diverging lenses or diverging deflective prisms.

6. The injector element (20) according to one of claims 1 to 5, wherein the light source (23) comprises a plurality of vertical-cavity surface-emitting laser (VCSEL) diodes, said plurality of diodes being arranged so as to form an emission surface (26) substantially perpendicular to the longitudinal axis (22) of the body (21).

7. The injector element (20) according to claim 6, wherein the light source comprises vertical-cavity surface-emitting laser (VCSEL) diodes all configured to emit light at substantially equal wavelengths.

8. The injector element (20) according to claim 7, wherein a phosphor (39) is applied against a side wall (24, 24b, 21b) of the body (21), the vertical-cavity surface-emitting laser (VCSEL) diodes being configured to emit light at wavelengths corresponding to blue light.

9. The injector element (20) according to claim 6, wherein the light source (23) comprises a first group of vertical-cavity surface-emitting laser (VCSEL) diodes configured to emit light at wavelengths corresponding to red light, a second group of vertical-cavity surface-emitting laser (VCSEL) diodes configured to emit light at wavelengths corresponding to blue light, and a third group of vertical-cavity surface-emitting laser (VCSEL) diodes configured to emit light at wavelengths corresponding to, such that the injector element (20) emits white light.

10. The injector element (20) according to one of claims 6 to 9, wherein the light source (23) is configured to emit more light in a peripheral zone (33) than in a central zone (34) of the emission surface (26).

11. The injector element (20) according to claim 10, wherein the light source (23) is configured to emit light only in the peripheral zone (33).

12. The injector element (20) according to one of claims 6 to 11, wherein the central zone (34) of the emission surface (26) contains no (VCSEL) diodes.

13. The injector element (20) according to one of claims 10 and 11, further comprising a control unit (29) configured to control the light source (23) such that the peripheral zone (33) of the emission surface (26) emits more light than the central zone (24).

14. The injector element (20) according to one of claims 10 to 13, wherein the light source (23) is configured to emit a non-uniform density of energy in the peripheral zone (33) of the emission surface (26).

15. The injector element (20) according to claim 14, wherein the (VCSEL) diodes each have an elementary emission surface, and wherein the elementary emission surfaces of the (VCSEL) diodes of the peripheral zone (33) have different dimensions such that the light source (23) emits non-uniform density of energy in the peripheral zone (33) of the emission surface (26).

16. The injector element (20) according to one of claims 14 and 15, further comprising power injectors (28) configured to deliver the (VCSEL) diodes a non-uniform density of electric current or voltage such that the light source (23) emits a non-uniform density of energy in the peripheral zone (33) of the emission surface (26).

17. The injector element (20) according to one of claims 10 to 16, wherein the optical elements (35i) are configured to deflect towards the outside of the body (21) all the light emitted by the peripheral zone (33) of the emission surface (26).

18. The injector element (20) according to one of claims 6 to 17, further comprising an end mirror (31) arranged at one end of the body (21) opposite the light source (23) so as to send back in the body (21) some of the light beam reflecting against said end mirror (31).

19. The injector element (20) according to one of claims 1 to 18, wherein the body (21) has a cylindrical form, in particular cylindrical in revolution or parallelepiped.

20. The injector element (20) according to claim 19, wherein the body (21) has the form of a cylinder of revolution.

21. The injector element (20) according to claim 20, wherein a mirror (41) is applied against a part of the body (21) corresponding to a half-cylinder, so as to reflect towards the inside of the body (21) the energy emitted towards said mirror.

22. The injector element (20) according to claim 19, wherein the body (21) has the form of a half-cylinder of revolution.

23. The injector element (20) according to claim 22, wherein a mirror (42) is applied against a planar side wall (24a) so as to reflect towards the inside of the body (21) the energy emitted towards said mirror.

24. The injector element (20) according to one of claims 6 to 9 in combination with one of claims 22 and 23, wherein the emission surface (26) of the light source (23) has the form of a half-disc, and wherein the light source (23) is configured to emit a quantity of light energy decreasing as it moves away from the straight line section (260) of the emission surface (26) in a direction (261) extending perpendicularly to said straight line section.

25. The injector element (20) according to claim 19, wherein the body (21) substantially has the form of a rectangular parallelepiped.

26. The injector element (20) according to claim 25, wherein the body (21) comprises a first plate (21a) and a second plate (21b), between which are placed at least one couple of optical elements (35i), the optical elements (35i) of each couple being placed facing and at a distance from each other so as to form an opening (38i) substantially coaxial with the longitudinal axis (22) of the body (21) so as to let through the fraction of the light beam spreading in the central part (36i) of the body (21).

27. The injector element (20) according to claim 25, wherein the body (21) comprises a plate (21b) and a plane mirror (43) placed facing each other, and at least one optical element (35i) placed at a distance from the plane mirror (43) so as to form an opening (38i) substantially coaxial with the longitudinal axis (22) of the body (21) so as to let through the fraction of the light beam spreading in the central part (36i) of the body (21).

28. A photobioreactor (10) intended for culture especially continuous culture of photosynthetic microorganisms, preferably microalgae, said photobioreactor (10) comprising at least one culture container (11) intended to contain the culture medium (12) of the microorganisms, said photobioreactor (10) being characterized in that it comprises a light injector element (20) according to one of claims 1 to 27, the body (21) of said injector element (20) being placed in the culture container (11).

29. A lighting element (50) for domestic lighting, characterized in that it comprises a light injector element (20) according to one of claims 1 to 27.

30. The lighting element (50) according to claim 29, further comprising a mirror (40) placed facing the body (21) so as to reflect the energy emitted towards said mirror.
